(19) United States
(12) Patent Application Publication
(10) Pub. No.: US 2026/0217590 A1
(43) Pub. Date: Jul. 30, 2026

(54) OPTICAL FIBER PREFORM MANUFACTURING METHOD AND OPTICAL FIBER PREFORM CHARACTERISTIC PREDICTION METHOD

(71) Applicant: SUMITOMO ELECTRIC INDUSTRIES, LTD., Osaka (JP)

(72) Inventors: Tatsuya KIMURA, Utsunomiya-shi (JP); Shigenori IITSUKA, Utsunomiya-shi (JP); Tomohiro ISHIHARA, Osaka (JP)

(21) Appl. No.: 19/462,006

(22) Filed: Jan. 28, 2026

(30) Foreign Application Priority Data
Jan. 29, 2025 (JP) 2025-013142

(51) Int. Cl. C03B 37/07; C03B 37/012

(52) U.S. Cl. CPC C03B 37/07; C03B 37/012

(57) ABSTRACT

An optical fiber preform manufacturing method includes a manufacturing step of manufacturing an optical fiber preform, a measurement step of measuring a refractive index distribution of the optical fiber preform, and a determination step of determining a quality of the optical fiber preform, based on a characteristic prediction result obtained by inputting, to a characteristic prediction model created in advance, a measurement result in the measurement step. The characteristic prediction model is a model obtained by machine learning using training data in which refractive index distribution data of an optical fiber preform manufactured in the past is used as an explanatory variable and a measurement result of a characteristic of an optical fiber obtained by drawing the optical fiber preform is used as an objective variable.

### CROSS-REFERENCE TO RELATED APPLICATIONS

This application is based on and claims priority under 35 USC 119 from Japanese Patent Application No. 2025-013142 filed on Jan. 29, 2025, the entire content of which is incorporated herein by reference.

### TECHNICAL FIELD

The present disclosure relates to an optical fiber preform manufacturing method and an optical fiber preform characteristic prediction method.

### BACKGROUND ART

JP2003-12337A discloses a method of predicting characteristics of an optical fiber obtained from an optical fiber preform by using a finite element method based on a refractive index of the preform.

### SUMMARY OF INVENTION

According to an aspect of the present disclosure, there is provided an optical fiber preform manufacturing method including:
a manufacturing step of manufacturing an optical fiber preform;
a measurement step of measuring a refractive index distribution of the optical fiber preform; and
a determination step of determining a quality of the optical fiber preform, based on a characteristic prediction result obtained by inputting, to a characteristic prediction model created in advance, a measurement result in the measurement step,
in which the characteristic prediction model is a model obtained by machine learning using training data in which refractive index distribution data of an optical fiber preform manufactured in the past is used as an explanatory variable and a measurement result of a characteristic of an optical fiber obtained by drawing the optical fiber preform is used as an objective variable.

### BRIEF DESCRIPTION OF DRAWINGS

Exemplary embodiment(s) of the present invention will be described in detail based on the following figures, wherein:

FIG. 1 is a block diagram illustrating an example of a manufacturing system for executing an optical fiber preform manufacturing method according to an embodiment of the present disclosure;

FIG. 2 is a flowchart illustrating the optical fiber preform manufacturing method according to the embodiment of the present disclosure;

FIG. 3 is a diagram illustrating an example of a refractive index distribution of a depressed structure; and

FIG. 4 is a diagram illustrating an example of a refractive index distribution of a trench structure.

### DESCRIPTION OF EMBODIMENTS

Standards are provided for various characteristics, such as a cutoff wavelength of an optical fiber, and an optical fiber whose characteristics do not satisfy the standards cannot be shipped. These characteristics vary depending on manufacturing or drawing conditions of the preform, and since it takes about several weeks from an upstream preform manufacturing step until the optical fiber is obtained, when manufacturing conditions are changed based on measured values of the characteristics of the manufactured optical fiber, it takes time until the change is reflected. Therefore, it is desirable to predict the characteristics of the optical fiber at the stage of the preform and adjust the manufacturing conditions based on prediction results. However, in methods in the related art, it is less likely to accurately predict the characteristics of the optical fiber at the stage of the preform.

An object of the present disclosure is to provide an optical fiber preform manufacturing method and a characteristic prediction method capable of accurately predicting a characteristic of an optical fiber based on a measurement result of an optical fiber preform.

According to the present disclosure, it is possible to accurately predict the characteristic of the optical fiber based on the measurement result of the optical fiber preform.

### DESCRIPTION OF EMBODIMENT OF PRESENT DISCLOSURE

First, aspects of the present disclosure will be listed and described. (1) An optical fiber preform manufacturing method according to the present disclosure in an optical fiber preform manufacturing method including: a manufacturing step of manufacturing an optical fiber preform; a measurement step of measuring a refractive index distribution of the optical fiber preform; and a determination step of determining a quality of the optical fiber preform based on a characteristic prediction result obtained by inputting, to a characteristic prediction model created in advance, a measurement result in the measurement step, in which the characteristic prediction model is a model obtained by machine learning using training data in which refractive index distribution data of an optical fiber preform manufactured in the past is used as an explanatory variable and a measurement result of a characteristic of an optical fiber obtained by drawing the optical fiber preform is used as an objective variable.

In the related art, a method of predicting the refractive index distribution of the optical fiber preform using the finite element method is known, but the prediction accuracy is not high. To improve the prediction accuracy, it is necessary to periodically perform correction or the like, which is a burden on an operator. According to the present embodiment, the characteristic of the optical fiber preform can be predicted with high accuracy by using a machine learning model created based on the data of the optical fiber preform manufactured in the past. Accordingly, the accuracy of the quality determination is improved, and the manufacturing efficiency can be improved.
(2) In the above (1), the characteristic prediction result may include data related to at least one characteristic selected from the group consisting of a cutoff wavelength, a mode field diameter, and a zero dispersion wavelength. By using the method of the present embodiment, the cutoff wavelength, the mode field diameter, or the zero dispersion wavelength can be predicted with high accuracy based on the refractive index distribution of the optical fiber preform.
(3) In the above (2), the characteristic prediction result may include the cutoff wavelength, and the characteristic prediction model may further include a drawing tension as the explanatory variable, and a cutoff wavelength corrected based on the drawing tension, as the objective variable. Since the cutoff wavelength varies depending on the drawing tension, the measured value is corrected based on the drawing tension, and by including the drawing tension as the explanatory variable, the corrected cutoff wavelength can be predicted.
(4) In the above (3), a manufacturing condition in a drawing step may be adjusted based on the characteristic prediction result of the optical fiber preform. By changing the drawing tension according to a value of the predicted cutoff wavelength, the cutoff wavelength of the optical fiber to be manufactured can be adjusted to a more appropriate range. By such adjustment, a range of the optical fiber preform usable for the product is widened, and manufacturing efficiency is improved.
(5) In any one of the above (1) to (4), the characteristic prediction model may further include, as the explanatory variable, a predicted value calculated by a finite element method based on the refractive index distribution for at least one of the objective variables. By adding the predicted value by the finite element method as the explanatory variable, the prediction accuracy is improved.
(6) In any one of the above (1) to (5), the refractive index distribution may be a depressed structure or a trench structure. In the related art, it is less likely to predict characteristics based on a complicated refractive index distribution such as a depressed structure or a trench structure. According to the present embodiment, by using the characteristic prediction model obtained by machine learning, the characteristic can be accurately predicted even in the optical fiber preform having a complicated refractive index distribution.
(7) In the above (6), the characteristic prediction model may further include, as the explanatory variable, a difference ΔN between a refractive index of a core and a refractive index of an inner cladding, in the refractive index distribution, and a depressed amount that is a difference between the refractive index of the inner cladding and a refractive index of an outer cladding, in the refractive index distribution. By adding the ΔN and the depressed amount as the explanatory variables, the prediction accuracy is improved.
(8) An optical fiber preform characteristic prediction method according to the present disclosure is a characteristic prediction method including a measurement step of measuring a refractive index distribution of an optical fiber preform, and a prediction step of predicting a characteristic of the optical fiber preform by inputting, to a characteristic prediction model created in advance, a measurement result in the measurement step, and the characteristic prediction model is a model obtained by machine learning using training data in which refractive index distribution data of an optical fiber preform manufactured in the past is used as the explanatory variable and the measurement result of the characteristic of the optical fiber obtained by drawing the optical fiber preform is used as an objective variable. According to the present embodiment, the characteristic of the optical fiber preform can be predicted with high accuracy by using a machine learning model created based on the data of the optical fiber preform manufactured in the past.

### DETAILS OF EMBODIMENT OF PRESENT DISCLOSURE

Specific examples of an optical fiber preform manufacturing method and a characteristic prediction method of the present disclosure will be described below with reference to the drawings. The present invention is not limited to these exemplifications, but is indicated by the scope of claims, and is intended to include all modifications within a scope and meaning equivalent to the scope of claims. At least a part of the embodiment described below may be freely combined.

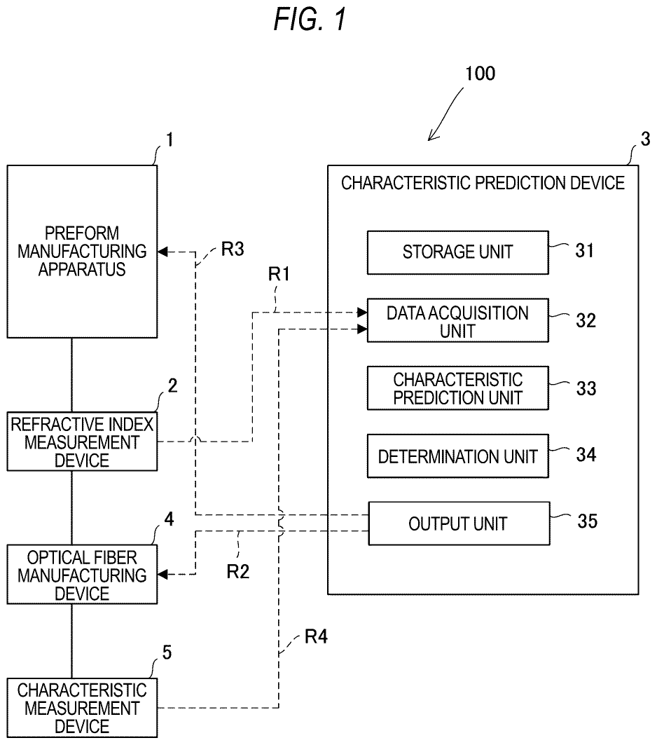

FIG. 1 is a block diagram illustrating an example of a manufacturing system for executing an optical fiber preform manufacturing method of the present disclosure. As illustrated in FIG. 1, the manufacturing system 100 includes a preform manufacturing apparatus 1, a refractive index measurement device 2, and a characteristic prediction device 3. FIG. 1 further illustrates an optical fiber manufacturing device 4 configured to draw an optical fiber preform manufactured by the preform manufacturing apparatus 1 to manufacture an optical fiber, and a characteristic measurement device 5 configured to measure a characteristic of the optical fiber manufactured by the optical fiber manufacturing device 4.

The preform manufacturing apparatus 1 is an apparatus for manufacturing the optical fiber preform. In the present disclosure, the optical fiber preform manufacturing method is not particularly limited, and for example, a vapor phase synthesis method such as a VAD method, an OVD method, or an MCVD method is adopted. The preform manufacturing apparatus 1 is not limited to a single apparatus and may include a plurality of devices. For example, the preform manufacturing apparatus 1 may include a device configured to manufacture a glass fine particle deposit by the vapor phase synthesis method exemplified above, and a consolidate furnace that consolidates the glass fine particle deposit to make the glass fine particle deposit transparent. In the preform manufacturing apparatus 1, various manufacturing conditions for manufacturing an optical fiber preform having desired characteristics are set. The manufacturing conditions of the preform manufacturing apparatus 1 may be adjusted by a control device (not illustrated). Parameters specifically adjusted as the manufacturing conditions may vary depending on the adopted method, and for example, an amount and a blending ratio of a raw material, a heating temperature, and a heating time May be adjusted. The optical fiber preform manufactured by the preform manufacturing apparatus 1 has a concentric structure including a core with a high refractive index and a cladding with a low refractive index that covers the core.

The refractive index measurement device 2 is configured to measure a refractive index of the optical fiber preform manufactured by the preform manufacturing apparatus 1. A known method can be adopted as a method of measuring the refractive index, and for example, a refractive index distribution along a diameter can be obtained by analyzing a transmitted light when a laser is emitted perpendicularly to an axial direction of the preform. The refractive index measurement device 2 may be provided in the preform manufacturing apparatus 1. The refractive index of one optical fiber preform may be measured at a plurality of positions along a longitudinal direction. When the refractive index is measured at the plurality of positions, all pieces of refractive index distribution data may be used in characteristic prediction to be described later, a part of the pieces of the refractive index distribution data may be used, or refractive index distribution data obtained by averaging a plurality of refractive index distributions may be used. A measurement position of the refractive index is preferably a central portion of the preform in the longitudinal direction, and specifically, is preferably a portion 50 mm or more inside an end portion of a region of the optical fiber preform to be drawn into a product.

The characteristic prediction device 3 is configured to predict the characteristic of the optical fiber obtained by drawing the optical fiber preform, based on the refractive index distribution data of the optical fiber preform measured by the refractive index measurement device 2. The characteristic prediction device 3 includes a storage unit 31, a data acquisition unit 32, a characteristic prediction unit 33, a determination unit 34, and an output unit 35. The characteristic prediction device 3 may be connected to another device via a cable, a network, or the like (not illustrated).

A hardware configuration of the characteristic prediction device 3 is not particularly limited. Each piece of processing (each function) of the embodiment to be described later is implemented by a circuitry including one or more processors. The circuitry may be implemented by an integrated circuit in which one or more memories, various analog circuits, various digital circuits are combined in addition to the one or more processors. The one or more memories store a program (an instruction) for causing the one or more processors to execute each piece of processing. The one or more processors may execute each piece of processing according to the program read from the one or more memories, or may execute each piece of processing according to a logic circuit designed to execute each piece of processing in advance. The processor may be various processors suitable for control of a computer, such as a central processing unit (CPU), a graphics processing unit (GPU), a digital signal processor (DSP), a field programmable gate array (FPGA), or an application specific integrated circuit (ASIC). The plurality of physically separated processors may cooperate with each other to execute each piece of processing. For example, the processor mounted on each of a plurality of physically separated computers may cooperate with each other via a network, such as a local area network (LAN), a wide area network (WAN), or the Internet, to execute each piece of processing. The program may be installed in the memory from an external server device via the network, or may be distributed in a state of being stored in a recording medium such as a compact disc read only memory (CD-ROM), a digital versatile disk read only memory (DVD-ROM), or a semiconductor memory, and installed in the memory from the recording medium.

The storage unit 31 is configured to store a program for executing processing of predicting the characteristic of the optical fiber and a model used for predicting the characteristic (hereinafter, referred to as a characteristic prediction model). A determination step S3 of determining a quality of the optical fiber preform to be described later is performed by, for example, the CPU executing the program stored in the storage unit 31. The characteristic prediction model is a model obtained by machine learning using training data in which refractive index distribution data of an optical fiber preform manufactured in the past is an explanatory variable and the measurement result of the characteristic of the optical fiber obtained by drawing the optical fiber preform is an objective variable. By inputting the refractive index distribution data of the optical fiber preform to the characteristic prediction model, a prediction result of the characteristic of the optical fiber obtained by drawing the optical fiber preform is output.

The characteristic of the optical fiber used as the objective variable of the characteristic prediction model is not particularly limited, and may be, for example, at least one or more characteristics selected from a cutoff wavelength, a mode field diameter, and a zero dispersion wavelength. The cutoff wavelength is the shortest wavelength propagating in the optical fiber in a single mode. The mode field diameter represents spread of a light propagating in the optical fiber in a radial direction. The zero dispersion wavelength is a wavelength at which a chromatic dispersion is zero.

A machine learning algorithm used to generate the characteristic prediction model is not particularly limited as long as the machine learning algorithm is classified into supervised learning, and may be a classification or regression method, such as a decision tree, a logistic regression analysis, or a multiple regression analysis, or ensemble learning in which a plurality of learners are combined. Specific examples of the algorithm include light gradient boosting machine (LightGBM), Ridge, extreme gradient boosting (XGBoost), and RandomForest.

The number of past data sets related to the optical fiber preform and the optical fiber used to generate the characteristic prediction model may be 5000 or more, or may be 20000 or more. When the number of data sets is 5000 or more, prediction accuracy is improved.

The data acquisition unit 32 is configured to acquire data input to the characteristic prediction device 3 from an outside. The characteristic prediction unit 33 is configured to predict the characteristic of the optical fiber, based on the data acquired by the data acquisition unit 32 and the characteristic prediction model. The determination unit 34 is configured to determine the quality of the optical fiber preform, based on the prediction result by the characteristic prediction unit 33. The output unit 35 is configured to output a determination result by the determination unit 34. Functions of the data acquisition unit 32, the characteristic prediction unit 33, the determination unit 34, and the output unit 35 will be described in detail in the description of the optical fiber preform manufacturing method to be described later.

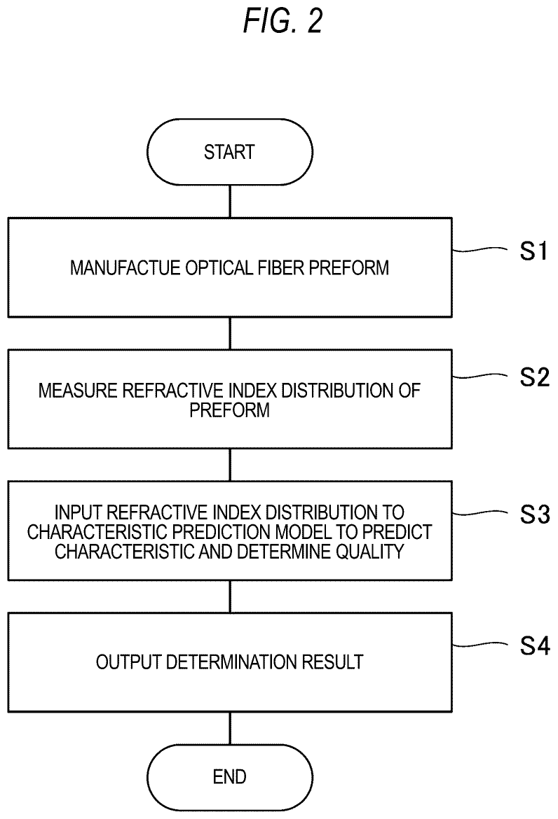

The optical fiber preform manufacturing method according to the embodiment of the present disclosure will be described. FIG. 2 is a flowchart of the optical fiber preform manufacturing method according to the present embodiment. As illustrated in FIG. 2, the optical fiber preform manufacturing method according to the present embodiment includes the manufacturing step S1, a measurement step S2, and a determination step S3. FIG. 2 further illustrates an output step S4 as a step after the determination step S3.

First, in the manufacturing step S1, the optical fiber preform is manufactured by the preform manufacturing apparatus 1. Next, in the measurement step S2, the refractive index of the optical fiber preform manufactured by the preform manufacturing apparatus 1 is measured by the refractive index measurement device 2 to acquire the refractive index distribution data. The refractive index distribution data obtained here is, for example, numerical data of the refractive index at a plurality of (for example, 1200) positions along a diameter of the optical fiber preform.

Next, in the determination step S3, the measurement result in the measurement step S2 is input to the characteristic prediction model created in advance to predict the characteristic of the optical fiber obtained by drawing the optical fiber preform, and the quality of the optical fiber preform is determined based on a characteristic prediction result. Specifically, first, the refractive index distribution data acquired by the refractive index measurement device 2 in the measurement step S2 is input to the data acquisition unit 32 of the characteristic prediction device 3 (an arrow R1 in FIG. 1). Next, the characteristic prediction unit 33 inputs the refractive index distribution data input to the data acquisition unit 32 to the characteristic prediction model stored in the storage unit 31, and obtains the characteristic prediction result of the optical fiber obtained from the optical fiber preform. The determination unit 34 determines the quality of the optical fiber preform based on the characteristic prediction result obtained by the characteristic prediction unit 33.

A method of determining the quality of the optical fiber preform in the determination unit 34 is not particularly limited, and, for example, the quality may be determined based on whether a predicted value of each characteristic calculated by the characteristic prediction model is included in a predetermined allowable range.

In the present embodiment, in the output step S4 after the determination step S3, the output unit 35 outputs the determination result by the determination unit 34. For example, the output unit 35 may display the determination result on an output device, such as a display, to notify an operator of the determination result. The output unit 35 may transmit the determination result or a command based on the determination result to the optical fiber manufacturing device 4, and manufacturing conditions of the optical fiber manufacturing device 4 may be changed based on the transmitted determination result or command (an arrow R2 in FIG. 1). The output unit 35 may transmit the determination result or the command based on the determination result to the preform manufacturing apparatus 1, and manufacturing conditions of the preform manufacturing apparatus 1 may be changed based on the transmitted determination result or command (an arrow R3 in FIG. 1).

In the related art, for example, the finite element method (FEM) has been used as a method of predicting the characteristic of the optical fiber obtained by drawing the optical fiber preform based on the characteristic of the optical fiber preform, such as the refractive index. However, in the finite element method, it is less likely to accurately predict the characteristic of the optical fiber at the stage of the preform.

According to the present embodiment, the refractive index distribution data of the optical fiber preform is input to the characteristic prediction model obtained by machine learning to predict the characteristic of the optical fiber and to determine the quality. By adopting such a configuration, the characteristic of the optical fiber can be accurately predicted at the stage of the preform.

When the characteristic prediction result includes the cutoff wavelength, in other words, when the objective variable includes the cutoff wavelength, the characteristic prediction model may further include drawing tension as the explanatory variable, and may further include the cutoff wavelength corrected based on the drawing tension as the objective variable. Since the cutoff wavelength of the optical fiber varies depending on the drawing tension, measured values can be corrected based on the drawing tension, and by including the drawing tension as the explanatory variable, the corrected cutoff wavelength can be predicted. When the drawing tension is included in the explanatory variable, since drawing is not performed at the time of characteristic prediction, each characteristic is predicted by inputting a temporary drawing tension.

When the characteristic prediction result includes the cutoff wavelength, manufacturing conditions in a drawing step may be adjusted based on the characteristic prediction result of the optical fiber preform. By changing the drawing tension according to a value of the predicted cutoff wavelength, the cutoff wavelength of the optical fiber to be manufactured can be adjusted to a more appropriate range. For example, when the drawing tension is increased, the cutoff wavelength of the manufactured optical fiber is large. Therefore, when a predicted value of the cutoff wavelength of the optical fiber preform is less than a target value, the cutoff wavelength of the manufactured optical fiber can be made larger than the predicted value by increasing the tension when drawing the optical fiber preform, and an optical fiber having desirable characteristics can be obtained. According to this configuration, even when the predicted cutoff wavelength does not satisfy the standard, the cutoff wavelength can be adjusted to satisfy the standard by changing the manufacturing conditions, such as the drawing tension, and therefore, a range of the optical fiber preform usable for the product is widened, and manufacturing efficiency is improved.

In the above description, “adjusting the manufacturing conditions in the drawing step” does not necessarily mean that actual drawing is performed under the manufacturing conditions. That is, the adjustment of the manufacturing conditions here is intended to calculate appropriate manufacturing conditions of the drawing step based on the characteristic prediction result and associate the calculated manufacturing conditions with the optical fiber preform. Therefore, for example, an aspect in which the calculated manufacturing conditions are added to the optical fiber preform when the optical fiber preform is shipped directly in a state of the preform is also included in the method according to the present embodiment.

The characteristic prediction model in the present embodiment may further include, as the explanatory variable, a predicted value calculated by the finite element method based on the refractive index distribution for at least one objective variable. For example, when the objective variable is the cutoff wavelength, the mode field diameter, and the zero dispersion wavelength of the optical fiber, a predicted value of at least one selected from the cutoff wavelength, the mode field diameter, and the zero dispersion wavelength may be calculated based on the refractive index distribution using the finite element method, and a model including the predicted value in the explanatory variable may be created. By adding the predicted value by the finite element method as the explanatory variable, the prediction accuracy is improved.

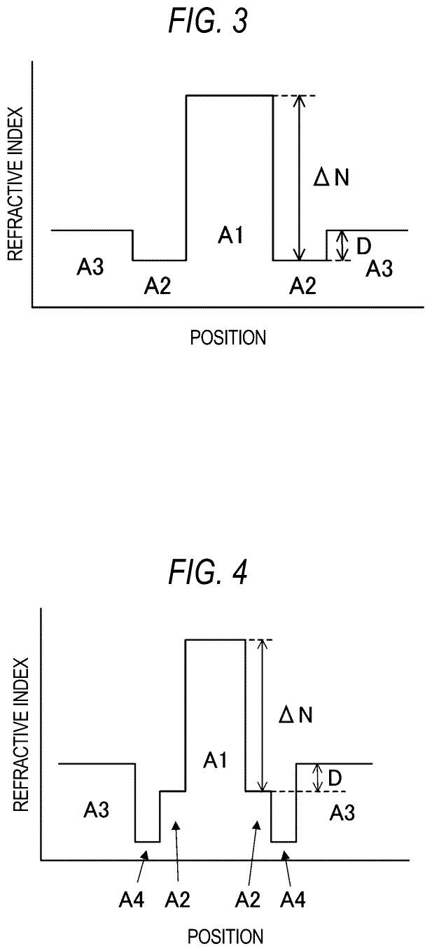

In the present embodiment, the refractive index distribution of the optical fiber preform may be a depressed structure or a trench structure. FIGS. 3 and 4 are graphs illustrating examples of the refractive index distribution, in which a horizontal axis represents a position in a direction along a diameter and a vertical axis represents the refractive index.

FIG. 3 illustrates a refractive index distribution of an optical fiber preform having a depressed structure. As illustrated in FIG. 3, in the depressed structure, a refractive index of an inner cladding A2 located adjacent to a core A1 in a cladding region is less than a refractive index of an outer cladding A3. FIG. 4 illustrates a refractive index distribution of an optical fiber preform having a trench structure. As illustrated in FIG. 4, in the trench structure, a low refractive index portion (a trench) A4 is provided between the inner cladding A2 and the outer cladding A3 in the cladding region.

In the related art, it is less likely to predict the characteristic of the optical fiber based on a complicated refractive index distribution, such as a depressed structure or a trench structure. According to the present embodiment, by using the characteristic prediction model obtained by machine learning, the characteristic of the optical fiber can be accurately predicted even in the optical fiber preform having a complicated refractive index distribution.

As illustrated in FIG. 3, in the depressed structure, a difference between the refractive index of the core A1 and the refractive index of the inner cladding A2 is referred to as ΔN, and a difference between the refractive index of the inner cladding A2 and the refractive index of the outer cladding A3 is referred to as a depressed amount D. As illustrated in FIG. 4, also in the trench structure, a difference between the refractive index of the core A1 and the refractive index of the inner cladding A2 is referred to as ΔN, and the difference between the refractive index of the inner cladding A2 and the refractive index of the outer cladding A3 is referred to as the depressed amount D. In the present embodiment, the characteristic prediction model may further include, as the explanatory variables, the difference ΔN between the refractive index of the core and the refractive index of the inner cladding in the refractive index distribution, and the depressed amount D, which is a difference between the refractive index of the inner cladding and the refractive index of the outer cladding. By adding the ΔN and the depressed amount D as the explanatory variables, the prediction accuracy is improved. The ΔN and the depressed amount D may be calculated by processing by a processor based on the refractive index distribution data, or values calculated after approximating the refractive index distribution to a stepwise distribution as illustrated in FIGS. 3 and 4 may be used.

In the present disclosure, the optical fiber may be manufactured by drawing the optical fiber preform manufactured by the preform manufacturing apparatus 1 by the optical fiber manufacturing device 4, the characteristic of the optical fiber may be measured by the characteristic measurement device 5, and then the measurement result may be input to the data acquisition unit 32 of the characteristic prediction device 3 (an arrow R4 in FIG. 1). A combination of the measurement result of the characteristic received from the characteristic measurement device 5 and the refractive index distribution data received from the refractive index measurement device 2 can be used as new training data. The characteristic prediction device 3 may create a new characteristic prediction model by adding, to the data set, data including the refractive index distribution data of the optical fiber preform obtained in this way and the characteristic measurement result of the optical fiber manufactured from the optical fiber preform, or may perform fine tuning of an existing characteristic prediction model. By creating or adjusting the characteristic prediction model using the data set of the sequentially manufactured optical fibers, the prediction accuracy of the characteristic prediction model can be improved.

The present disclosure also relates to an optical fiber preform characteristic prediction method. The characteristic prediction method includes the measurement step of measuring the refractive index distribution of the optical fiber preform, and a prediction step of predicting the characteristic of the optical fiber preform by inputting the measurement result in the measurement step to the characteristic prediction model created in advance, and the characteristic prediction model is a model obtained by machine learning using training data in which refractive index distribution data of an optical fiber preform manufactured in the past is used as the explanatory variable and the measurement result of the characteristic of the optical fiber obtained by drawing the optical fiber preform is used as an objective variable.

According to the method of the present embodiment, the characteristic of the optical fiber preform can be predicted with high accuracy, and the prediction result can be used for quality assurance, verification, and the like of the optical fiber preform.

### EXAMPLE

A characteristic prediction model was created by a machine learning algorithm based on about 20000 data sets including the refractive index distribution data of the optical fiber preform manufactured in the past and the characteristic of the optical fiber obtained by drawing the optical fiber preform. As the machine learning algorithm, LightGBM, Ridge, XGBoost, and RandomForest were used. The explanatory variable of the characteristic prediction model was the refractive index distribution data (one-dimensional refractive index data at about 1200 positions along the diameter) of the optical fiber preform, and the objective variable was the cutoff wavelength, the mode field diameter (MFD), and the zero dispersion wavelength. Using the obtained characteristic prediction model, each characteristic as the objective variable was predicted based on the refractive index distribution data in the 2000 evaluation data sets, and a double root mean square error (RMSE) was calculated based on the predicted value and the measured value. Results are illustrated in Table 1.

For comparison, characteristic prediction was performed for the same evaluation data sets using the finite element method (FEM), and the RMSE of each objective variable was calculated. Results are illustrated in Table 1. Table 1 illustrates a relative value of the RMSE of each characteristic prediction model when the RMSE in the case of the finite element method is 1.

TABLE 1

| | | | | | Random |
|---|---|---|---|---|---|
| Characteristics | FEM | LightGBM | Ridge | XGBoost | Forest |
| Cutoff wavelength | 1 | 0.52 | 0.62 | 0.55 | 0.53 |
| MFD | 1 | 0.33 | 0.37 | 0.35 | 0.36 |
| Zero dispersion | 1 | 0.12 | 0.34 | 0.14 | 0.16 |
| wavelength | | | | | |

As illustrated in Table 1, it can be seen that in any example using the characteristic prediction model generated by machine learning, the RMSE is less than that with the finite element method, and the prediction accuracy is improved.

What is claimed is:

1. An optical fiber preform manufacturing method comprising:
a manufacturing step of manufacturing an optical fiber preform;
a measurement step of measuring a refractive index distribution of the optical fiber preform; and
a determination step of determining a quality of the optical fiber preform, based on a characteristic prediction result obtained by inputting, to a characteristic prediction model created in advance, a measurement result in the measurement step,
wherein the characteristic prediction model is a model obtained by machine learning using training data in which refractive index distribution data of an optical fiber preform manufactured in the past is used as an explanatory variable and a measurement result of a characteristic of an optical fiber obtained by drawing the optical fiber preform is used as an objective variable.

2. The optical fiber preform manufacturing method according to claim 1,
wherein the characteristic prediction result includes data related to at least one characteristic selected from the group consisting of a cutoff wavelength, a mode field diameter, and a zero dispersion wavelength.

3. The optical fiber preform manufacturing method according to claim 2,
wherein the characteristic prediction result includes the cutoff wavelength, and
the characteristic prediction model further includes:
a drawing tension, as the explanatory variable; and
a cutoff wavelength corrected based on the drawing tension, as the objective variable.

4. The optical fiber preform manufacturing method according to claim 3,
wherein a manufacturing condition in a drawing step is adjusted based on the characteristic prediction result of the optical fiber preform.

5. The optical fiber preform manufacturing method according to claim 1,
wherein the characteristic prediction model further includes, as the explanatory variable, a predicted value calculated by a finite element method based on the refractive index distribution for at least one of the objective variables.

6. The optical fiber preform manufacturing method according to claim 1,
wherein the refractive index distribution is a depressed structure or a trench structure.

7. The optical fiber preform manufacturing method according to claim 6,
wherein the characteristic prediction model further includes, as the explanatory variable:
a difference ΔN between a refractive index of a core and a refractive index of an inner cladding, in the refractive index distribution; and
a depressed amount that is a difference between the refractive index of the inner cladding and a refractive index of an outer cladding, in the refractive index distribution.

8. An optical fiber preform characteristic prediction method comprising:
a measurement step of measuring a refractive index distribution of an optical fiber preform; and
a prediction step of predicting a characteristic of the optical fiber preform by inputting, to a characteristic prediction model created in advance, a measurement result in the measurement step,
wherein the characteristic prediction model is a model obtained by machine learning using training data in which refractive index distribution data of an optical fiber preform manufactured in the past is used as an explanatory variable and a measurement result of a characteristic of an optical fiber obtained by drawing the optical fiber preform is used as an objective variable.
